(19) United States
(12) Patent Application Publication
(10) Pub. No.: US 2022/0249551 A1
(43) Pub. Date: Aug. 11, 2022

(54) STIMULATION OF NATURAL KILL CELL MEMORY BY ADMINISTRATION OF DENDRITIC CELLS

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); James VELTMEYER, Oceanside, CA (US); Famela RAMOS, Oceanside, CA (US)

(73) Assignee: Therapeutic Solutions International, Inc., Oceanside, CA (US)

(21) Appl. No.: 17/667,349

(22) Filed: Feb. 8, 2022

Related U.S. Application Data
(60) Provisional application No. 63/146,999, filed on Feb. 8, 2021.

(51) Int. Cl. A61K 35/15; A61P 35/00; A61K 38/17

(52) U.S. Cl. CPC A61K 35/15; A61K 38/1774; A61P 35/00

(57) ABSTRACT

Disclosed are means, methods and compositions of matter useful for induction of natural killer cell memory by administration of dendritic cells and/or exosomes thereof. In one embodiment a mammal suffering from cancer is administered allogeneic cord blood derived dendritic cells that are not pulsed exogenously. In one embodiment the dendritic cells are stimulated to possess chemotactic activity towards the tumor by culture of dendritic cell progenitors in hypoxia. Natural killer cell memory is induced, in part, by triggering of upregulation of cytokines associated with homeostatic expansion such as interleukin 7 and interleukin 15.

### CROSS-REFERENCE TO RELATED APPLICATIONS

This application claims priority to U.S. Provisional Application Ser. No. 63/146,999, filed on Feb. 8, 2021, which is incorporated by reference in its entirety.

### FIELD OF THE INVENTION

The invention belongs to the area of immune modulation, more specifically the invention belongs to the field of stimulating immunity by modulating natural killer cell memory by administration of dendritic cells and/or exosomes thereof. These methods are useful for treating cancer.

### BACKGROUND

New and useful treatments of immune modulation and particular treatments of cancer are needed in the art.

### SUMMARY

Preferred embodiments include methods of inducing natural killer cell memory in a host suffering from cancer comprising the steps of: a) obtaining a patient suffering from an oncological disease; b) administering to said patient a population of dendritic cells in a manner in which said dendritic cells interact with said tumor in said patient; and c) optionally providing agents which induce stimulation of NK cell memory formation.

Preferred methods include embodiments wherein said dendritic cells are allogeneic to the recipient.

Preferred methods include embodiments wherein said dendritic cells are autologous to the recipient.

Preferred methods include embodiments wherein said dendritic cells are xenogeneic to the recipient.

Preferred methods include embodiments wherein said dendritic cells are obtained from bone marrow.

Preferred methods include embodiments wherein said dendritic cells are obtained from pluripotent stem cells.

Preferred methods include embodiments wherein said dendritic cells are obtained from mobilized peripheral blood.

Preferred methods include embodiments, wherein said dendritic cells are obtained from placenta.

Preferred methods include embodiments wherein said dendritic cells are obtained from umbilical cord blood.

Preferred methods include embodiments wherein said dendritic cells are obtained from CD133 cells.

Preferred methods include embodiments wherein said dendritic cells are obtained from CD34 cells.

Preferred methods include embodiments wherein said dendritic cells are obtained from alternatively activated macrophages.

Preferred methods include embodiments wherein said dendritic cells are cultured under hypoxia before administration.

Preferred methods include embodiments wherein said dendritic cells are cultured under acidosis before administration.

Preferred methods include embodiments wherein said dendritic cells are cultured under hypo-osmotic conditions before administration.

Preferred methods include embodiments wherein said dendritic cells are cultured under hyper-osmotic conditions before administration.

Preferred methods include embodiments wherein said dendritic cells are cultured under hypothermia or hyperthermia before administration.

Preferred methods include embodiments wherein said dendritic cells are cultured in the presence of a toll like receptor agonist before administration.

Preferred methods include embodiments wherein said toll like receptor is toll like receptor 1.

Preferred methods include embodiments wherein said TLR-1 is activated by Pam3CSK4.

Preferred methods include embodiments wherein said immune receptor is TLR-2

Preferred methods include embodiments wherein said TLR-2 is activated by HKLM.

Preferred methods include embodiments wherein said immune receptor is TLR-3.

Preferred methods include embodiments wherein said TLR-3 is activated by Poly:IC.

Preferred methods include embodiments wherein said immune receptor is TLR-4.

Preferred methods include embodiments wherein said TLR-4 is activated by LPS.

Preferred methods include embodiments wherein said TLR-4 is activated by Buprenorphine.

Preferred methods include embodiments wherein said TLR-4 is activated by Carbamazepine.

Preferred methods include embodiments wherein said TLR-4 is activated by Fentanyl.

Preferred methods include embodiments wherein said TLR-4 is activated by Levorphanol.

Preferred methods include embodiments wherein said TLR-4 is activated by Methadone.

Preferred methods include embodiments wherein said TLR-4 is activated by Cocaine.

Preferred methods include embodiments wherein said TLR-4 is activated by Morphine.

Preferred methods include embodiments wherein said TLR-4 is activated by Oxcarbazepine.

Preferred methods include embodiments wherein said TLR-4 is activated by Oxycodone.

Preferred methods include embodiments wherein said TLR-4 is activated by Pethidine.

Preferred methods include embodiments wherein said TLR-4 is activated by Glucuronoxylomannan from Cryptococcus.

Preferred methods include embodiments wherein said TLR-4 is activated by Morphine-3-glucuronide.

Preferred methods include embodiments wherein said TLR-4 is activated by lipoteichoic acid.

Preferred methods include embodiments wherein said TLR-4 is activated by beta.-defensin 2.

Preferred methods include embodiments wherein said TLR-4 is activated by low molecular weight hyaluronic acid.

Preferred methods include embodiments wherein said low molecular weight hyaluronic acid has a molecular weight of <1000 kDa.

Preferred methods include embodiments wherein said low molecular weight hyaluronic acid has a molecular weight of <500 kDa.

Preferred methods include embodiments wherein said low molecular weight hyaluronic acid has a molecular weight of <250 kDa.

Preferred methods include embodiments wherein said low molecular weight hyaluronic acid has a molecular weight of <100 kDa.

Preferred methods include embodiments wherein said TLR-4 is activated by fibronectin EDA.

Preferred methods include embodiments wherein said TLR-4 is activated by snapin.

Preferred methods include embodiments wherein said TLR-4 is activated by tenascin C.

Preferred methods include embodiments wherein said immune receptor is TLR-5.

Preferred methods include embodiments wherein said TLR-5 is activated by flaggelin.

Preferred methods include embodiments wherein said immune receptor is TLR-6.

Preferred methods include embodiments wherein said TLR-6 is activated by FSL-1.

Preferred methods include embodiments wherein said immune receptor is TLR-7.

Preferred methods include embodiments wherein said TLR-7 is activated by imiquimod.

Preferred methods include embodiments wherein said immune receptor is TLR-8.

Preferred methods include embodiments wherein said TLR-8 is activated by ssRNA40/LyoVec.

Preferred methods include embodiments wherein said immune receptor is TLR-9.

Preferred methods include embodiments wherein said TLR-9 is activated by a CpG oligonucleotide.

Preferred methods include embodiments wherein said TLR-9 is activated by ODN2006.

Preferred methods include embodiments wherein said TLR-9 is activated by Agatolimod.

Preferred methods include embodiments wherein said TLR-9 is activated by ODN2007.

Preferred methods include embodiments wherein said TLR-9 is activated by ODN1668.

Preferred methods include embodiments wherein said TLR-9 is activated by ODN1826.

Preferred methods include embodiments wherein said TLR-9 is activated by ODN BW006.

Preferred methods include embodiments wherein said TLR-9 is activated by ODN DSL01.

Preferred methods include embodiments wherein said TLR-9 is activated by ODN 2395.

Preferred methods include embodiments wherein said TLR-9 is activated by ODN M362.

Preferred methods include embodiments wherein said TLR-9 is activated by ODN SL03.

Preferred methods include embodiments wherein said dendritic cells interact with said tumor due to intratumoral administration of said dendritic cells.

Preferred methods include embodiments wherein said tumor undergoes a therapy resulting in immunogenic cell death of one or more parts of said tumor prior to and/or concurrent with administration of said dendritic cells.

Preferred methods include embodiments wherein said therapy resulting in said immunogeneic cell death is exposure to one or more oncolytic viruses.

Preferred methods include embodiments wherein said therapy resulting in said immunogeneic cell death is exposure to one or more chemotherapeutic agents.

Preferred methods include embodiments wherein said therapy resulting in said immunogeneic cell death is exposure to one or more antiangiogenic agents.

Preferred methods include embodiments wherein said oncolytic virus is new castle disease virus.

Preferred methods include embodiments wherein said oncolytic virus is parvovirus.

Preferred methods include embodiments wherein said oncolytic virus is measles virus

Preferred methods include embodiments wherein said oncolytic virus is reovirus.

Preferred methods include embodiments wherein said oncolytic virus is vesicular stomatitis virus (VSV).

Preferred methods include embodiments wherein said oncolytic virus is adenovirus.

Preferred methods include embodiments wherein said oncolytic virus is poliovirus.

Preferred methods include embodiments wherein said oncolytic virus is herpes simplex virus (HSV).

Preferred methods include embodiments wherein said oncolytic virus is a poxvirus.

Preferred methods include embodiments wherein said oncolytic virus is coxsackie virus (CXV) and

Preferred methods include embodiments wherein said Seneca Valley virus (SVV).

Preferred methods include embodiments wherein said dendritic cell is generated by culture of a hematopoietic stem cells for a period of at least 1 hour.

Preferred methods include embodiments wherein said hematopoietic stem cell is capable of generating leukocytic, lymphocytic, thrombocytic and erythrocytic cells when transplanted into an immunodeficient animal.

Preferred methods include embodiments wherein said hematopoietic stem cell expresses interleukin-3 receptor.

Preferred methods include embodiments wherein said hematopoietic stem cell expresses interleukin-1 receptor.

Preferred methods include embodiments wherein said hematopoietic stem cell expresses c-met.

Preferred methods include embodiments wherein said hematopoietic stem cell expresses mpl.

Preferred methods include embodiments wherein said hematopoietic stem cell expresses interleukin-11 receptor.

Preferred methods include embodiments wherein said hematopoietic stem cell expresses G-CSF receptor.

Preferred methods include embodiments wherein said hematopoietic stem cell expresses GM-CSF receptor.

Preferred methods include embodiments wherein said hematopoietic stem cell expresses M-CSF receptor.

Preferred methods include embodiments wherein said hematopoietic stem cell expresses VEGF-receptor.

Preferred methods include embodiments wherein said hematopoietic stem cell expresses c-kit.

Preferred methods include embodiments wherein said hematopoietic stem cell expresses CD33.

Preferred methods include embodiments wherein said hematopoietic stem cell expresses CD133.

Preferred methods include embodiments wherein said hematopoietic stem cell expresses CD34.

Preferred methods include embodiments wherein said hematopoietic stem cell expresses Fas ligand.

Preferred methods include embodiments wherein said hematopoietic stem cell does not express lineage markers.

Preferred methods include embodiments wherein said hematopoietic stem cell does not express CD14.

Preferred methods include embodiments wherein said hematopoietic stem cell does not express CD16.

Preferred methods include embodiments wherein said hematopoietic stem cell does not express CD3.

Preferred methods include embodiments wherein said hematopoietic stem cell does not express CD56.

Preferred methods include embodiments wherein said hematopoietic stem cell does not express CD38.

Preferred methods include embodiments wherein said hematopoietic stem cell does not express CD30.

### BRIEF DESCRIPTION OF THE DRAWINGS

FIG. 1 is a graph showing tumor suppression based on cell type.

### DETAILED DESCRIPTION OF THE INVENTION

The invention provides means of treatment of cancer by inducing generation of natural killer cells which possess an immunological memory towards cancer. Specifically the invention reports that NK cells are capable of attaining immunological memory as a result of being exposed to allogeneic dendritic cells under conditions of not being pulsed.

“Adjuvant” refers to a substance that is capable of enhancing, accelerating, or prolonging an immune response when given with a vaccine immunogen.

“Agonist” refers to is a substance which promotes (induces, causes, enhances or increases) the activity of another molecule or a receptor. The term agonist encompasses substances which bind receptor (e.g., an antibody, a homolog of a natural ligand from another species) and substances which promote receptor function without binding thereto (e.g., by activating an associated protein).

“Antagonist” or “inhibitor” refers to a substance that partially or fully blocks, inhibits, or neutralizes a biological activity of another molecule or receptor.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells.

The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention.

Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma.

In the context of the present invention the term “culturing” refers to the in vitro propagation of cells or organisms in media of various kinds. It is understood that the descendants of a cell grown in culture may not be completely identical (morphologically, genetically, or phenotypically) to the parent cell. A suitable culturing medium can be selected by the person skilled in the art and examples of such media are RPMI medium or Eagles Minimal Essential Medium (EMEM).

The terms “vaccine”, “immunogen”, or immunogenic composition” are used herein to refer to a compound or composition that is capable of conferring a degree of specific immunity when administered to a human or animal subject. As used in this disclosure, a “cellular vaccine” or “cellular immunogen” refers to a composition comprising at least one cell population, which is optionally inactivated, as an active ingredient. The immunogens, and immunogenic compositions of this invention are active, which mean that they are capable of stimulating a specific immunological response (such as an anti-tumor antigen or anti-cancer cell response) mediated at least in part by the immune system of the host. The immunological response may comprise antibodies, immunoreactive cells (such as helper/inducer or cytotoxic cells), or any combination thereof, and is preferably directed towards an antigen that is present on a tumor towards which the treatment is directed. The response may be elicited or restimulated in a subject by administration of either single or multiple doses.

A compound or composition is “immunogenic” if it is capable of either: a) generating an immune response against an antigen (such as a tumor antigen) in a naive individual; or b) reconstituting, boosting, or maintaining an immune response in an individual beyond what would occur if the compound or composition was not administered. A composition is immunogenic if it is capable of attaining either of these criteria when administered in single or multiple doses.

The term “T-cell response” means the specific proliferation and activation of effector functions induced by a peptide in vitro or in vivo. For MHC class I restricted cytotoxic T cells, effector functions may be lysis of peptide-pulsed, peptide-precursor pulsed or naturally peptide-presenting target cells, secretion of cytokines, preferably Interferon-gamma, TNF-alpha, or IL-2 induced by peptide, secretion of effector molecules, preferably granzymes or perforins induced by peptide, or degranulation.

The term “peptide” is used herein to designate a series of amino acid residues, connected one to the other typically by peptide bonds between the alpha-amino and carbonyl groups of the adjacent amino acids. The peptides are preferably 9 amino acids in length but can be as short as 8 amino acids in length, and as long as 10, 11, 12, or even longer, and in case of MHC class II peptides (e.g. elongated variants of the peptides of the invention) they can be as long as 15, 16, 17, 18, 19, 20 or 23 or more amino acids in length.

Furthermore, the term “peptide” shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoro-acetate) salts. It has to be noted that the salts of the peptides according to the present invention differ substantially from the peptides in their state(s) in vivo, as the peptides are not salts in vivo.

In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-gamma, transforming growth factor (TGF)-beta, IL-15, IL-7, IFN-alpha, IL-12, CD40L, and IL-27.

From the viewpoint of enhancing cellular immunity, particularly suitably, IL-2, IFN-gamma, or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method.

In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects The invention provides means of utilizing dendritic progenitor cells and products derived from dendritic progenitor cells as a cancer vaccine which selectively induces immunity towards tumor vasculature and not healthy, non-malignant, vasculature. In one embodiment the invention teaches the utilization of culture conditions which mimic the tumor microenvironment as a means of creating a cellular population that resembles tumor endothelial cells. Culture conditions include the growth of endothelial progenitor cells in acidic conditions which resemble the tumor microenvironment. Numerous papers have characterized the acidic conditions in the tumor microenvironment and are incorporated by reference.

Interestingly, tumor acidic conditions are believed to be associated with resistance to immunotherapy. In a recent study it was shown that an acidic pH environment blocked T-cell activation and limited glycolysis in vitro. IFN.gamma. release blocked by acidic pH did not occur at the level of steady-state mRNA, implying that the effect of acidity was posttranslational. Acidification did not affect cytoplasmic pH, suggesting that signals transduced by external acidity were likely mediated by specific acid-sensing receptors, four of which are expressed by T cells. Notably, neutralizing tumor acidity with bicarbonate monotherapy impaired the growth of some cancer types in mice where it was associated with increased T-cell infiltration. Furthermore, combining bicarbonate therapy with anti-CTLA-4, anti-PD1, or adoptive T-cell transfer improved antitumor responses in multiple models, including cures in some subjects. In one embodiment of the invention, endothelial progenitor cells, or products thereof, are cultured under conditions in GCN2 kinase is activated, the conditions include culture in the presence of uncharged tRNA, tryptophan deprivation, arginine deprivation, asparagine deprivation [60-64], and glutamine deprivation.

In one embodiment the invention provides a means of generating an allogeneic population of cells with tumoricidal ability. 50 ml of peripheral blood is extracted from a cancer patient and peripheral blood monoclear cells (PBMC) are isolated using the Ficoll Method. PBMC are subsequently resuspended in 10 ml STEM-34 media and allowed to adhere onto a plastic surface for 2-4 hours. The adherent cells are then cultured at 37.degree. C. in STEM-34 media supplemented with 1,000 U/mL granulocyte-monocyte colony-stimulating factor and 500 U/mL IL-4 after non-adherent cells are removed by gentle washing in Hanks Buffered Saline Solution (HB SS). Half of the volume of the GM-CSF and IL-4 supplemented media is changed every other day. Immature DCs are harvested on day 7. In one embodiment the generated DC are used to stimulate T cell and NK cell tumoricidal activity. Specifically, generated DC may be further purified from culture through use of flow cytometry sorting or magnetic activated cell sorting (MACS) or may be utilized as a semi-pure population. DC may be added into the patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to the T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. In some embodiments the starting population of cells is umbilical cord blood cells. In specific embodiments said cells are umbilical cord blood CD34 cells.

In one embodiment of the invention, dendritic cells are used to carry oncolytic viruses into the tumor. The invention teaches delivery of an oncolytic virus to a subject having cancer, wherein the carrier cell is allogeneic to the subject, the method comprising: identifying one or more of the following determinants (a)-(f) as indicative of a match between the carrier cell and the subject: a) the carrier cell and the subject have identical alleles at 50% or more of the following genetic loci combined: (i) MHC I and/or MHC II haplotypes; (ii) KIR haplotype and/or KIR ligand haplotypes; and (iii) HLA-E, CD1a, CD1b, CD1c and/or CD1d haplotypes; b) incubating the carrier cell in a co-culture with cancerous cells from the subject results in one or more of the following: (i) a cell to tumor migration score (CTMS) of 20% or more of the carrier cells migrating toward the cancerous cells; (ii) a tumor to cell migration score (TCMS) of 20% or more of the cancerous cells migrating toward the carrier cells; and/or (iii) a cumulative migration score (MRS) of [(i)+(ii)]/2 of at least 20%; c) incubating the carrier cell in a co-culture with the oncolytic virus and cancerous cells from the subject results in one or more of the following: (i) a virus loaded cell to tumor migration score (V-CTMS) of 20% or more of the carrier cells migrating toward the cancerous cells; (ii) a virus loaded tumor to cell migration score (V-TCMS) of 20% or more of the cancerous cells migrating toward the carrier cells; and/or (iii) a cumulative virus loaded migration score (V-MRS) of [(i)+(ii)]/2 of at least 20%; d) when the carrier cell is incubated in a co-culture with the oncolytic virus and immune cells obtained from the subject, an immunological viral amplification score (IVAS) representing the amount of viral amplification in the presence of immune cells obtained from the subject relative to the amount of viral amplification obtained under equivalent conditions except in the absence of immune cells obtained from the subject, is at least 20%; e) when the carrier cell is incubated in a co-culture with the oncolytic virus and immune cells obtained from the subject, an immunological compatibility score (ICS) representing the immune response in the presence of the carrier cell relative to the immune response under equivalent conditions except in the absence of the carrier cell, is.ltoreq.200%, wherein the immune response determined by the amount of expression of one or more of the following: (i) IFN.gamma.; (ii) one or more markers associated with T cell, NK cell and/or NKT cell-mediated cytotoxicity; and/or (iii) one or more markers associated with T cell, NK cell and/or NKT cell activator/effector function(s); f) when the carrier cell is incubated in a co-culture with the oncolytic virus and immune cells obtained from the subject, the carrier cell does not augment an anti-viral immune response and/or suppresses an anti-viral immune response relative to identical conditions except in the absence of the carrier cell, as measured by an immunological suppression score (ISS) of .gtoreq.0% according to the equation: ISS %=[(IV+IC)−ICV]/(IV+IC).times.0.100, wherein: IV=the marker expression level in a co-culture of the virus+immune cells obtained from the subject; IC=the marker expression level in a co-culture of immune cells obtained from the subject+the carrier cell; ICV=the marker expression level in a co-culture of immune cells obtained from the subject+the carrier cell+the virus; and the marker expression level is the expression level of one or more of the markers set forth in (i), (ii) and (iii) of e); if one or more of a)-f) is satisfied, identifying a match between the carrier cell and the subject; and selecting the carrier cell as suitable for delivery of an oncolytic virus to the subject having cancer. This is one method in which the current inventions is to be practiced.

The oncolytic virus can be any known to those of skill in the art. Included are oncolytic viruses selected from among new castle disease virus, parvovirus, measles virus, reovirus, vesicular stomatitis virus (VSV), adenovirus, poliovirus, herpes simplex virus (HSV), poxvirus, coxsackie virus (CXV) and Seneca Valley virus (SVV). In some embodiments, the oncolytic virus is a vaccinia virus, such as, but not limited to, a smallpox vaccine. Exemplary vaccinia viruses include those derived from a Lister strain, Western Reserve (WR) strain, Copenhagen (Cop) strain, Bern strain, Paris strain, Tashkent strain, Tian Tan strain, Wyeth strain (DRYVAX), IHD-J strain, IHD-W strain, Brighton strain, Ankara strain, CVA382 strain, Dairen I strain, LC16m8 strain, LC16M0 strain, modified vaccinia Ankara (MVA) strain, ACAM strain, WR 65-16 strain, Connaught strain, New York City Board of Health (NYCBH) strain, EM-63 strain, NYVAC strain, Lister strain LIVP, JX-594 strain, GL-ONC1 strain, and vvDD TK mutant strain with deletions in VGF and TK (see, e.g., McCart et al. (2001) Cancer Res. 61:8751-8757). For example, the vaccinia virus can be ACAM2000 or ACAM1000. The viruses can be oncolytic adenovirus, such as, for example, ONYX-015, CG00070, Oncorin (H101), ColoAd1, ONCOS-102, or Delta24-RGD/DNX-2401. The virus can be a modified HSV-1 virus, or a measles virus. The oncolytic viruses can be modified to express a heterologous gene product and/or to have increased tumorigenicity and/or to have reduced toxicity (increased attenuation). The viruses can encode a detectable marker for detection in culture or in a subject. For example, the marker can be a fluorescent protein, such as Turbo-Red or GFP. Within the practice of the invention, it can be sensitized or treated, or engineered, to achieve, enhance or improve (compared to virus not sensitized, treated or engineered) one or more of: virus amplification in the cell, blocking the induction of an anti-viral state in a subject or in the tumor microenvironment, immune suppression/immune evasion, protection against allogeneic inactivation/rejection determinants, and/or protection against complement. Provided are carrier cells sensitized or engineered to enhance or improve virus amplification; or sensitized or treated or engineered to block induction of an anti-viral state in the subject or in the tumor microenvironment; or treated, sensitized or engineered to enhance virus amplification by pre-treatment or treatment with one or more of a cytokine or growth factor. For example, the carrier cells can be treated to inhibit, or modified to express an inhibitor of, interferon-.gamma. and/or interferon-.beta. In some embodiments, instead of treating the cells with an agent, the agent and cell can be co-administered to the host. Exemplary of dendritic carrier cells sensitized to enhance virus amplification, are those sensitized by pre-treatment or treatment to load the cell with one or more of IL-10, TGF.beta., VEGF, FGF-2, PDGF, HGF, IL-6, GM-CSF, a RTK/mTOR agonist, a Wnt protein ligand, and a GSK3 inhibitor/antagonist. Exemplary of carrier cells that have been sensitized to block induction of an anti-viral state are those sensitized by pre-treatment or treatment to load the cell with one or more small molecule or protein inhibitors that interfere with IFN Type I/Type II receptors, interfere with downstream IFN signaling, interfere with IFNAR1/IFNAR2 signaling, interfere with IFNGR1/IFNGR2 signaling, interfere with STAT1/2 signaling, interfere with Jak1 signaling, interfere with Jak2 signaling, interfere with IRF3 signaling, interfere with IRF7 signaling, interfere with IRF9 signaling, interfere with TYK2 signaling, interfere with TBK1 signaling, or interfere with other signaling pathways that effect an immune response against the oncolytic virus in the cell or subject. Exemplary of cells sensitized to block induction of an anti-viral state, are those sensitized by pre-treatment or treatment to load the cell with one or more HDAC inhibitors, for interfering with/deregulating IFN signaling/responsiveness. Exemplary HDAC inhibitors include, but are not limited to, those selected from among vorinostat, romidepsin, chidamide, panobinostat, belinostat, valproic acid, mocetinostat, abexinostat, entinostat, SB939, resminostat, givinostat, quisinostat, HBI-8000, Kevetrin, CUDC-101, AR-42, CHR-2845, CHR-3996, 4SC-202, CG200745, ACY-1215, ME-344, sulforaphane, or trichostatin. Exemplary of carrier cells sensitized to block induction of an anti-viral state or enhance virus amplification are those sensitized by pre-treatment or treatment to load the cell with antagonists of virus sensing and/or anti-virus defense pathways. Virus sensing and/or defense pathway(s) include those that is/are induced or modulated by one or more of STING, PKR, RIG-1, MDA-5, OAS-1/2/3, AIM2, MAVS, RIP-1/3, and DAI (ZBP1). Antagonists that affect one or more of these pathways include, for example, one or more of K1, E3L, and K3L vaccinia proteins; NS1/NS2 influenza proteins; hepatitis C NS3-4A; arenavirus NP and Z proteins; Ebola virus VP35; HSV US11, ICP34.5 and ICP0; MCMV M45; and Borna disease virus X protein.

Exemplary of dendritic cells that have been sensitized to protect them against inactivation/rejection determinants, are those sensitized by pre-treatment or treatment to load the cell with one or more viral major histocompatibility (MHC) antagonists. Exemplary MHC antagonists include those selected from among one or more of A40R MHCI antagonist from vaccinia; Nef and TAT from HIV; E3-19K from adenovirus; ICP47 from HSV 1 and HSV2; CPXV012 and CPXV203 from Cowpox; ORF66 from varicella zoster virus (VZV); EBNA1, BNLF2a, BGLF5, and BILF1 from Epstein Barr virus (EBV); US2/gp24, US3/gp23, US6/gp21, US10, and US11/gp33 from human cytomegalovirus (hCMV); Rh178/VIHCE from rhesus CMV (RhCMV); U21 from human herpes virus-6 (HHV6) or HHV7; LANAI, ORF37/SOX, kK3/MIR1, and kK5/MIR2 from Kaposi's sarcoma associated herpes virus (KSHV); mK3 from mouse hepatitis virus-68 (MHV-68); UL41/vhs from alpha-herpesvirus, herpes simplex virus (HSV), bovine herpes virus-2 (BHV-1), and pseudorabies virus (PRV); UL49.5 from Varicellovirus, BHV-1, equine herpes virus 1 and 4 (EHV-1/4) and PRV; and m4/gp34, m6/gp48, m27, m152/gp40 from murine CMV (mCMV). Other antagonists for pre-treatment or treatment are those that are treated to load the cell with antagonists of human MEW class I chain related genes MIC-A and MIC-B or with beta-2 microglobulin (B2M) antagonists of viral origin. Prevention of the cell therapy for being killed by comoplement is necessary. For example, by pre-treatment or treatment to load the cell with an antibody or small molecule or other inhibitor of a complement protein. Complement proteins that can be targeted include C3 and C5. As described above and below, and exemplified herein, there are numerous known inhibitors of C3 and C5, including antagonist antibodies specific for each, and small molecule inhibitors. Dendritic cells generally are pre-treated with the agent that sensitizes or protects. Pre-treatment can be effected for 15 min to 48 hours before viral infection, after viral infection, or before administration to the subject, or before storage. The dendritic cells carrier cells also can be sensitized or engineered for extended survival and improved local immunosuppression to reduce or limit virus-mediated killing. Agents for effecting this include agonist(s), such as of one or more of STING, PKR, RIG-I, MDA-5, OAS-1/2/3, AIM2, MAVS, RIP-1/3, and DAI (ZBP1), which can be engineered for expression by the cell or virus under control of a promoter that appropriately times expression, or by administration to the subject, so that the carrier cells are not killed too soon by virus, but are not killed or inhibited by the host's immune system before delivering virus to the tumors. The dendritic cells provided herein also can be engineered for improved viral amplification and/or immunomodulation and/or NK activation. This can be effected, for example, by one or more of: a) engineering to prevent or to be unresponsive to an interferon-induced antiviral state; b) engineering to evade allogeneic recognition by one or more of T and NKT cells and/or adaptive immune responses of .gamma..delta. T cells; c) engineering to evade allogeneic recognition by NK Cells and/or innate immune responses of .gamma..delta. T cells; d) engineering to express immunosuppressive factors of human or viral origin to prevent/inhibit allogeneic anti-carrier cell or anti-viral immune responses; e) engineering to express cancer or stem cell-derived factors that facilitate viral infection of otherwise non-permissive carrier cells and/or tumor cells; and f) engineering to express factors interfering with the function of complement and/or neutralizing antibodies.

### Example

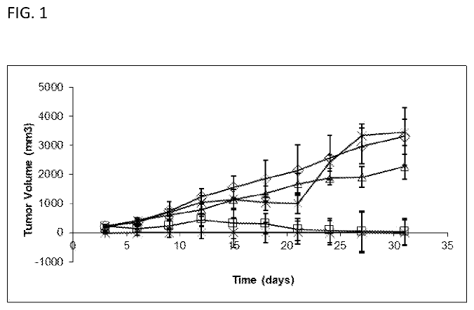

C57/BL6 mice were implanted with 500000 B16 melanoma cells and allowed to grow. StemVacs (human umbilical cord blood dendritic cells pulsed with poly IC (1 ug/ml for 4 hours) were administered to mice 5 days after tumor inoculation. The concentration of dendritic cells was 250,000 per mouse administrated intratumorally. Mice were observed until tumors regressed and given another 1 month after last tumor regression. Mice where then sacrificed and cells from transferred to naïve mice immunized with B16 melanoma. Cells were transferred 5 days after inoculation of B16. With reference to the graph in FIG. 1, Control (diamond), CD8 (X) and CD4 (triangle) all had no protection. In contrast transfer of splenocytes (Square) or CD56 cells (X) resulted in suppression of tumor growth.

1. A method of inducing natural killer cell memory in a host suffering from cancer comprising the steps of: a) obtaining a patient suffering from an oncological disease; b) administering to said patient a population of dendritic cells in a manner in which said dendritic cells interact with said tumor in said patient; and c) optionally providing agents which induce stimulation of NK cell memory formation.

2. The method of claim 1, wherein said dendritic cells are allogeneic to the recipient.

3. The method of claim 1, wherein said dendritic cells are autologous to the recipient.

4. The method of claim 1, wherein said dendritic cells are xenogeneic to the recipient.

5. The method of claim 1, wherein said dendritic cells are obtained from bone marrow.

6. The method of claim 1, wherein said dendritic cells are obtained from pluripotent stem cells.

7. The method of claim 1, wherein said dendritic cells are obtained from mobilized peripheral blood.

8. The method of claim 1, wherein said dendritic cells are obtained from placenta.

9. The method of claim 1, wherein said dendritic cells are obtained from umbilical cord blood.

10. The method of claim 1, wherein said dendritic cells are obtained from CD133 cells.

11. The method of claim 1, wherein said dendritic cells are obtained from CD34 cells.

12. The method of claim 1, wherein said dendritic cells are obtained from alternatively activated macrophages.

13. The method of claim 1, wherein said dendritic cells are cultured under hypoxia before administration.

14. The method of claim 1, wherein said dendritic cells are cultured under acidosis before administration.

15. The method of claim 1, wherein said dendritic cells are cultured under hypo-osmotic conditions before administration.

16. The method of claim 1, wherein said dendritic cells are cultured under hyper-osmotic conditions before administration.

17. The method of claim 1, wherein said dendritic cells are cultured under hypothermia or hyperthermia before administration.

18. The method of claim 1, wherein said dendritic cells are cultured in the presence of a toll like receptor agonist before administration.

19. The method of claim 1, wherein said NK cells express CD56

20. The method of claim 1, wherein said NK cells are derived from a pluripotent stem cell.
